(19) United States
(12) Patent Application Publication
(10) Pub. No.: US 2013/0123770 A1
(43) Pub. Date: May 16, 2013

(54) ABLATION CATHETER WITH CRYOTHERMAL BALLOON

(71) Applicant: Boston Scientific Scimed, Inc., Maple Grove, MN (US)

(72) Inventor: SCOTT R. SMITH, CHASKA, MN (US)

(73) Assignee: BOSTON SCIENTIFIC SCIMED, INC., MAPLE GROVE, MN (US)

(21) Appl. No.: 13/677,011

(22) Filed: Nov. 14, 2012

Related U.S. Application Data
(60) Provisional application No. 61/559,524, filed on Nov. 14, 2011.

(51) Int. Cl. A61B 18/08

(52) U.S. Cl. CPC A61B 18/082
USPC 606/28

(57) ABSTRACT

A catheter for thermal modulation of renal nerves via thermal ablation, the catheter including a catheter shaft including a distal end, a proximal end, and two lumens—a first lumen and a second lumen—extending therethrough. A thermal heating element is located proximate the distal end of the catheter shaft. In addition, a phase-change cooling mechanism is configured to extract heat away from the thermal heating element. The phase-change cooling mechanism involves vaporization of a liquid refrigerant circulated to the distal end of the catheter shaft through the first lumen into a gas and exhausting the gas to the proximal end of the catheter shaft through the second lumen.

### CROSS-REFERENCE TO RELATED APPLICATIONS

This application claims priority under 35 U.S.C. §119 to U.S. Provisional Application Ser. No. 61/559,524, filed Nov. 14, 2011, the entirety of which is incorporated herein by reference.

### TECHNICAL FIELD

This disclosure relates to devices and methods for intravascular neuromodulation. More particularly, the technologies disclosed herein relate to apparatus, systems, and methods for achieving intravascular renal neuromodulation via thermal heating.

### BACKGROUND

Certain treatments require the temporary or permanent interruption or modification of select nerve function. One example of such a treatment is renal nerve ablation, which is sometimes used to treat conditions related to congestive heart failure. The kidneys produce a sympathetic response to congestive heart failure, which, among other effects, increases the undesired retention of water and/or sodium. Ablating some of the nerves running to the kidneys may reduce or eliminate this sympathetic function, which may provide a corresponding reduction in the associated undesired symptoms.

Many nerves (and nervous tissue such as brain tissue), including renal nerves, run along the walls of or in close proximity to blood vessels, and thus can be accessed intravascularly through the walls of the blood vessels. In some instances, it may be desirable to ablate perivascular renal nerves using a radio frequency (RF) electrode. However, such a treatment may result in thermal injury to the vessel wall at the electrode, and other undesirable side effects, such as, but not limited to, blood damage, clotting and/or protein fouling of the electrode.

It is therefore desirable to provide for alternative systems and methods for intravascular nerve modulation.

### SUMMARY

The disclosure is directed to several alternative designs, and methods of using medical device structures and assemblies for performing nerve ablation.

Accordingly, one illustrative embodiment is a catheter for thermal modulation of renal nerves via thermal ablation. The catheter includes a shaft including a distal end, a proximal end, and first and second lumens extending therethrough. A thermal heating element is located proximate the distal end of the catheter shaft. In addition, a phase-change cooling mechanism is configured to extract heat away from the thermal heating element. The phase-change cooling mechanism involves vaporization of a liquid refrigerant circulated to the distal end of the catheter shaft through the first lumen and exhausting the resulting gas to the proximal end of the catheter shaft through the second lumen.

Some embodiments pertain to a method of intravascularly thermally modulating renal nerve tissue. The method includes advancing a catheter through a vessel such that a distal end of the catheter is positioned proximate a target renal nerve location.

Subsequently, activating a thermal heating element mounted on the distal end of the catheter to thermally heat a renal nerve tissue. In addition, the method includes cooling the thermal heating element with a phase-change cooling mechanism. The cooling mechanism includes steps of delivering a pressurized liquid refrigerant through the catheter to an expansion chamber proximate the thermal heating element, expanding the pressurized liquid refrigerant into a gas to absorb heat generated from the thermal heating element, and exhausting the gas from the expansion chamber.

The above summary of some example embodiments is not intended to describe each disclosed embodiment or every implementation of the present disclosure.

### BRIEF DESCRIPTION OF THE DRAWINGS

The invention may be more completely understood in consideration of the following detailed description of various embodiments in connection with the accompanying drawings, in which:

FIG. 1 is a schematic view illustrating a renal nerve modulation system in situ.

FIG. 2 is a side view of an exemplary cryothermal ablation system received in a blood vessel.

FIG. 3 is a side view of an alternate embodiment of the cryothermal ablation system shown in FIG. 2.

FIG. 4 is a side view of another embodiment of the cryothermal ablation system shown in FIG. 2.

FIG. 5 is a cross-sectional view of an embodiment of a cryothermal ablation catheter device.

FIG. 6 is a cross-sectional view of another embodiment of a cryothermal ablation catheter device.

FIG. 7 is a cross-sectional view of yet another embodiment of a cryothermal ablation catheter device.

While the invention is amenable to various modifications and alternative forms, specifics thereof have been shown by way of example in the drawings and will be described in detail. It should be understood, however, that the intention is not to limit aspects of the invention to the particular embodiments described. On the contrary, the intention is to cover all modifications, equivalents, and alternatives falling within the spirit and scope of the invention.

### DETAILED DESCRIPTION

For the following defined terms, these definitions shall be applied, unless a different definition is given in the claims or elsewhere in this specification. All numeric values are herein assumed to be modified by the term “about”, whether or not explicitly indicated. The term “about” generally refers to a range of numbers that one of skill in the art would consider equivalent to the recited value (i.e., having the same function or result). In many instances, the term “about” may be indicative as including numbers that are rounded to the nearest significant figure.

The recitation of numerical ranges by endpoints includes all numbers within that range (e.g., 1 to 5 includes 1, 1.5, 2, 2.75, 3, 3.80, 4, and 5).

Although some suitable dimensions ranges and/or values pertaining to various components, features and/or specifications are disclosed, one of skill in the art, incited by the present disclosure, would understand desired dimensions, ranges and/or values may deviate from those expressly disclosed.

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise.

The following detailed description should be read with reference to the drawings in which similar elements in different drawings are numbered the same. The detailed description and the drawings, which are not necessarily to scale, depict illustrative embodiments and are not intended to limit the scope of the invention. The illustrative embodiments depicted are intended only as exemplary. Selected features of any illustrative embodiment may be incorporated into an additional embodiment unless clearly stated to the contrary.

While the devices and methods described herein are discussed relative to ablation of perivascular renal nerves for treatment of hypertension, it is contemplated that the devices and methods may be used in other applications where nerve modulation and/or ablation are desired.

The present disclosure provides methods and systems to ablate a renal nerve. To this end, the system may employ a catheter having both an ultrasound energy source and a phase-change cooling mechanism, including an expansion chamber disposed at the distal end of the catheter. An ultrasound transducer, disposed on the surface of the chamber or close thereto, may emit ultrasound energy to ablate the perivascular renal nerves. During this process an inlet lumen extending between the chamber and the proximal end of the catheter may provide a flow of refrigerant fluid, which vaporizes upon introduction to the chamber. The phase change of the fluid absorbs considerable heat, and the resulting gas may be removed though an exhaust lumen. Thus, the phase-change cooling mechanism may provide active cooling to avoid thermal damage and/or prolonged cooling of the ultrasound transducer and/or surrounding tissue/blood during ablation.

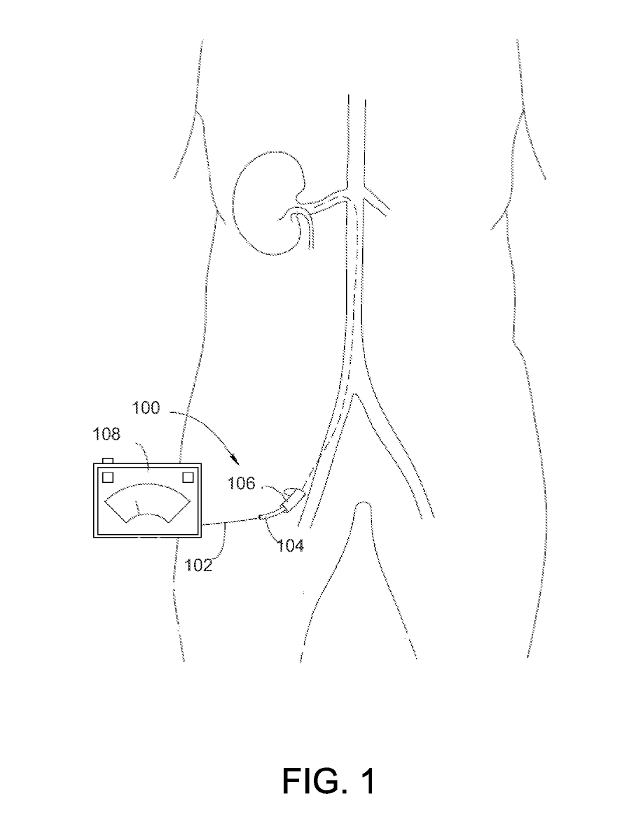

FIG. 1 is a schematic view of an illustrative renal nerve modulation system 100 in situ. System 100 may include one or more conductive element(s) 102 providing power to a renal ablation system 104 disposed within a sheath 106, the details of which can be better seen in subsequent figures.

A proximal end of conductive element 102 may be connected to a control and power element 108, which supplies the electrical energy to activate the one or more electrodes (e.g., radio frequency electrodes) or ultrasound transducers at or near a distal end of the renal ablation system 104. The control and power element 108 may include monitoring elements to monitor parameters such as power, temperature, voltage, frequency, pulse size and/or shape and other suitable parameters as well as suitable controls for performing the desired procedure. The power element 108 may control an ultrasound transducer to operate at a desired ultrasound energy range. The renal ablation system 104 employed by the present disclosure may employ a phase-change cryothermal cooling mechanism that allows the ultrasound transducers to be used at higher power for a prolonged period of time for efficient nerve ablation. In addition, the cooling mechanism may help reduce pain, provide tissue numbing or mild hypothermia, and/or avoid thermal tissue damage. The phase-change cryothermal cooling mechanism may function according to the Joule-Thomson effect, which describes the temperature change of a gas or liquid when it is forced through an orifice or valve, known as a throttling process. According to the Joule-Thomson effect, the fluid will cool upon expansion across the orifice or valve.

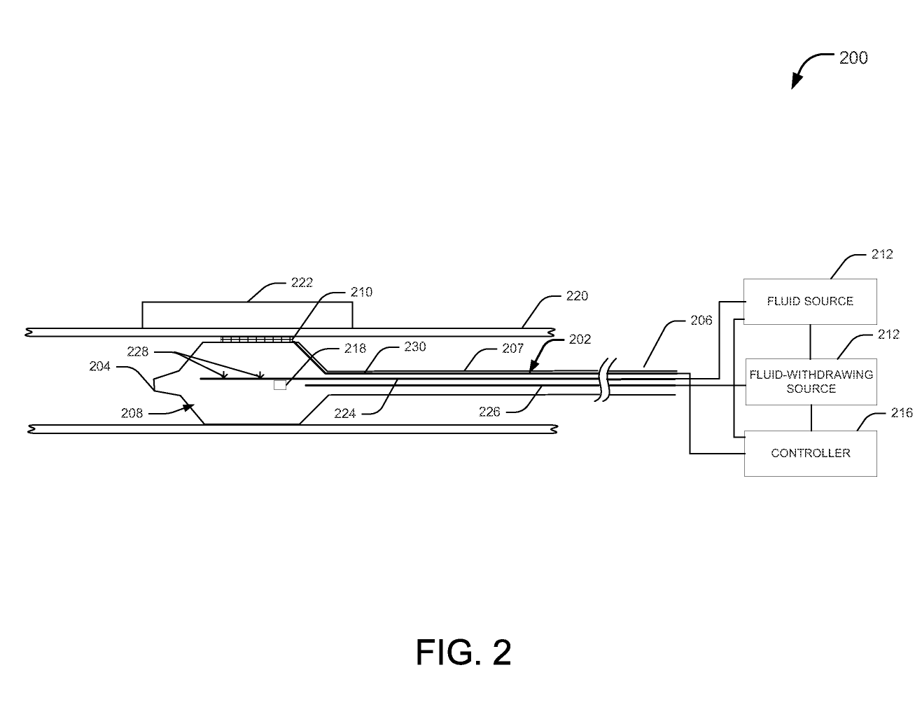

FIG. 2 illustrates a side view of an exemplary embodiment of a cryoablation system 200 configured for use in the renal nerve modulation system 100 depicted in FIG. 1. The cryoablation system 200 may include an elongated catheter 202 having a distal end 204, a proximal end 206, and an elongated shaft 207 extending from the proximal end 206 to the distal end 204. In the illustrated embodiment, the cryoablation system 200 may be configured to implement a phase-change cryothermal cooling mechanism by using an inflatable balloon 208 coupled to the distal end 204 of the catheter 202 as an expansion chamber. For ablation purposes, an ultrasound transducer 210 may be mounted on the exterior surface of the balloon 208 or on a portion of the catheter shaft 207 or distal tip proximate the balloon 208. In other embodiments, the cryoablation system 200 may include an RF electrode or other thermal element configured to ablate tissue, such as nerve tissue. In addition, a fluid source 212, a fluid-withdrawing source 214, and a controller 216 may be coupled to the proximal end 206 of the catheter 202. In at least some embodiments, one or more sensors 218 located proximate the distal end 204 of the catheter 202 which are connected to the controller 216 or other monitoring device may monitor one or more conditions (e.g., pressure or temperature) proximate the distal end 204 of the catheter 202, such as within the catheter 202, within the balloon 208, or the temperature of the blood and/or luminal surface of the blood vessel proximate the site of ablation.

Catheter 202 may be configured to advance into a body lumen having a vessel wall 220 (e.g., a renal artery) to ablate a body tissue 222 (e.g., renal nerves or ganglia). Catheter 202 may be formed of a hollow shaft having a cross-sectional configuration adapted to be received in a desired body lumen, such as a renal artery. Furthermore, the catheter shaft 207 may include a plurality of lumens extending therethrough. For example, the catheter shaft 207 may include a guidewire lumen configured to permit the catheter 202 to be advanced over a guidewire. The catheter shaft 207 may also include fluid delivery and exhaust lumens as further described herein. However, depending upon the particular implementation and intended use, the configuration of catheter 202 may vary. For instance, the catheter 202 may have a sufficient length such that the distal end 204 may extend into the body lumen while the proximal end 206 remains outside a patient's body. In addition, the catheter 202 may taper at its distal end to provide convenient insertion in a patient's body and/or navigation through the vasculature.

In the embodiment illustrated in FIG. 2, the balloon 208 may be sized to substantially circumferentially contact the luminal surface of the blood vessel upon inflation of the balloon 208 within the vessel. In other embodiments, the distal portion of the catheter 202 may be bent at a desired angle, directing towards the target tissue 222. To this end, in some embodiments the catheter 202 may be fabricated with the distal portion being bent at a predetermined angle such that the distal end portion automatically reverts to the pre-formed bent shape when unconstrained and/or when the balloon 208 is inflated, for example. In an alternate embodiment, the catheter 202 may include a steering mechanism (not shown) to manually bend the distal portion at a desired angle once the catheter 202 is positioned close to the tissue 222. For example, pull wires connected to the distal end of the catheter 202 may extend up to the proximal end 206 and a user may manipulate these wires to bend or straighten the distal portion, as desired.

Balloon 208 may define a cooling region of the catheter 202 such as defining an expansion chamber for the fluid. The balloon 208 may be short, less than a few centimeters long, for example, and be located proximate the distal tip of the catheter 202 or slightly proximal to the distal end 204. In other embodiments, the balloon 208 may have a longer length, if desired. In addition, balloon 208 may be an integral part of the catheter 202 or a separate component connected to the distal end of the catheter 202, such as by adhesive bonding or thermal bonding, for example. The balloon 208 may be a chamber expandable in nature attributable to the material composition of the chamber. Some exemplary suitable materials include polymers, including elastomeric materials. Examples of suitable polymeric materials include, but are not limited to, polyethylene terephthalate (PET), polytetrafluoroethylene (PTFE), polyurethane, polyamide (nylon), polyether, polyether block amide (PEBA), or other suitable materials, mixtures, combinations or copolymers thereof.

Balloon 208 may be in fluid communication with the fluid source 212 and fluid-withdrawing source 214 through a set of lumens extending through the catheter 202. To this end, the catheter 202 may include an inlet lumen 224 and an exhaust lumen 226, each in fluid communication with the interior of the balloon 208, to facilitate continuous flow of fluid to and from the balloon 208, respectively. These lumens may be generally elongated hollow tubes extending completely or partially along the entire length of the elongated shaft 207, or these lumens may be defined in the elongated shaft 207, such as during an extrusion process. The proximal ends of the inlet and exhaust lumens 224, 226 may be connected to the fluid source 212 and the fluid-withdrawing source 214, respectively. In some instances, the fluid being exhausted from the exhaust lumen 226 may be recirculated through the fluid source 212 to the inlet lumen 224, to provide a closed fluid pathway of the cooling system. Depending upon the particular implementation and intended use, the number and configuration of lumens may vary.

In general, fluid may be delivered to balloon 208 at relatively high operating pressure, which may be in the range of about 30 to 200 psig in some instances. In addition, the pressure at which fluid is supplied to the catheter 202 at a catheter inlet may range from about 100 to 1000 psig in some instances, depending on flow rate, flow resistance and the thermodynamic properties of the fluid. The distal end of the inlet lumen 224 may include expansion mechanisms to facilitate expansion of the injected fluid as the fluid is expelled into the balloon 208. For example, the distal end of the inlet lumen 224 may include or be formed as one or more orifices 228 to accomplish the fluid delivery to the balloon 208. The orifices 228 may release the pressurized fluid within the balloon 208, whereupon the phase-changing fluid vaporizes into gas, and thus changes phase. The injection of fluid through the orifice 228 may also result in substantial drop in fluid pressure in the range of about 200 to 600 psi. It is evident that the presence or absence of orifices 228, the number of orifices 228 and their orientation on the inlet lumen 224 may vary, with the configuration of the orifice(s) 228 outputting the fluid within the balloon 208. Alternatives to the expansion mechanism include valves, nozzles, or other known devices. Exemplary cooling effects of this fluid expansion cycle are discussed below.

Ultrasound transducer 210, which may be attached to the outer surface of the balloon 208, may be configured as a heating element adapted to ablate the target tissue. The transducer 210 can be configured as a disc, a plate, a strut, a ring, a cuboid, or other suitable configuration, as desired. In addition, an ablation source (such as the control and power element 108 shown in FIG. 1), may be located at the proximal end of the catheter 202 to provide power to the transducer 210 through connectors 230, such as wires. Ultrasound transducer 210 may be adapted to deliver high energy ultrasound into a localized region to ablate tissue. Although the illustrated embodiment depicts ultrasound transducer 210 as a source of ablation energy, it is envisioned than any of a number of energy sources can be employed. Suitable sources of ablation energy include, but are not limited to, radio frequency (RF) energy, microwaves, photonic energy, and thermal energy.

In the embodiment illustrated in FIG. 2, the transducer 210 may be positioned on the balloon 208, such that inflation of the balloon 208 urges the transducer 210 against the vessel wall. However, in other embodiments, the balloon 208 and/or transducer 210 may be configured and arranged such that when the balloon 208 is inflated and in circumferential contact with the vessel wall, the transducer 210 is spaced away from the vessel wall in an off-the-wall configuration. For example, the balloon 208 may be formed with a recessed portion within which the transducer 210 may be mounted. Thus, the outer surface of the transducer 210 may be located radially inward from the outer radial extent of the balloon 208. Accordingly, when the outer radial extent of the balloon 208 contacts the vessel wall upon inflation, the transducer 210 may remain spaced away from the vessel wall. In such an embodiment, the balloon 208 may be a non-distensible balloon which retains a preformed shape when inflated.

Controller 216 may control the flow of fluid within the catheter 202 to and from the balloon 208. To this end, the controller 216 may be connected both to the fluid source 212 and the fluid-withdrawing source 214, which may include a fluid reservoir and pump and/or suction device, respectively. The controller 216 may operate to facilitate a continuous flow of fluid to the balloon 208 through the inlet lumen 224 and from the balloon 208 through the exhaust lumen 226. In some embodiments, the controller 216 may also be connected to the sensor 218 to monitor desired conditions proximate the distal end of the catheter 202. For example, the sensor 218 may measure the temperature of the fluid, blood and/or luminal vessel wall to identify whether the cooling level is sufficient, or whether to increase or decrease the cooling level.

Fluid source 212 can provide a variety of cryogenic fluids, as desired. For example, the cryoablation system 200 may employ a phase-change liquid that can vaporize to gas upon expansion in the expansion chamber. This phase-change fluid may be stored in the fluid source 212 at ambient temperature but relatively high pressure. Suitable phase-change liquids include nitrous oxide, Freon, argon, liquid nitrogen, liquid propane, chlorofluorocarbon (CFC), hydrochlorofluorocarbon (HCFC), hydrofluorocarbon (HFC), or similar compounds. In some embodiments, liquid coolants, such as cold saline, ethyl alcohol, an ice slurry, or other chilled liquid, that do not undergo a phase change may also be used.

The fluid in general may act as a coolant or refrigerant that cools the outer surface of the balloon 208 by extracting heat energy away from the balloon 208 and surrounding blood and/or tissue. Specifically, cooling can be achieved by injecting high-pressure coolant carried within the inlet lumen 224 through one or more orifices 228. Upon ejection from the orifices 228, the coolant enters a relatively low-pressure environment, whose conditions of pressure and temperature are chosen to cause the fluid to vaporize immediately. This phase change absorbs a considerable quantity of heat, cooling the surface of the balloon 208 and surrounding blood and/or tissue. In one embodiment, the fluid may be pre cooled to lower its pressure and increase cooling capacity prior to reaching the expansion chamber, e.g., the balloon 208. By operating the phase-change cooling mechanism continuously during the ablation procedure, the vascular tissue surrounding the balloon can be maintained at a desired temperature and/or a temperature providing a therapeutic effect.

The cooled outer surface of the balloon 208 can both cool the blood and/or tissue proximate to the balloon 208 and cool the ultrasound transducer 210. As transducer 210 dissipates a substantial portion of its ultrasound energy as heat, cooling the transducer may be useful to avoid damaging the vessel walls 220 and/or may prolong the time available to ablate the tissue. In addition, in some instances it may be desirable to initially numb the target tissue (e.g., reduce the temperature of the target tissue to a mild hypothermic condition) before ablating the tissue with the ultrasound energy from the transducer 210. The balloon 208 proximate the target tissue may accomplish this task by cooling the tissue close to its freezing temperature or otherwise induce mild hypothermia. The cooling balloon 208 of the present disclosure may provide focused cooling at a site adjacent the balloon 208, avoiding excessive cooling of adjacent tissues.

The expansion of the balloon 208 may also assist in pushing the transducer 210 against the vessel walls 220 to provide effective ablation. For example, the diameter of the balloon 208 may be sized such that when the balloon 208 is fully inflated within the lumen of the vessel consequent the fluid flow through the balloon 208, the balloon 208 may substantially circumferentially contact the luminal surface of the vessel wall, and thus urging the transducer 210 against the luminal surface of the vessel wall. In other embodiments, expansion of the balloon 208 may position the transducer 210 at a predetermined spaced relationship with the vessel wall, in which the transducer 210 remains spaced from the vessel wall.

Several alternative embodiments of the cryoablation system 200 may be contemplated. For example, in some embodiments a liquid coolant, not employing the phase change technique, may suffice to effect sufficient cooling. Such embodiments may employ a simple liquid coolant, instead of the phase-change mechanism and refrigerant described above.

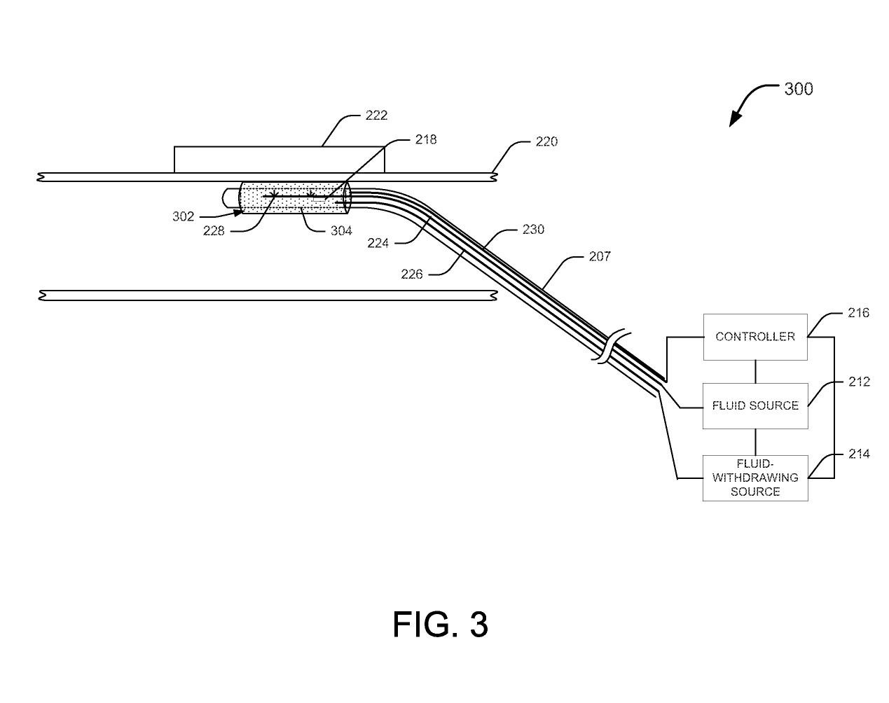

FIG. 3 illustrates an embodiment of a cryoablation system 300 including an ultrasound transducer configured as a hollow cylindrical transducer 302 that is fitted over a distal portion of the catheter 202. In one aspect, cylindrical transducer 302 may be configured to be disposed around an expandable chamber 304 disposed at a distal portion of the catheter 202. Accordingly, the dimensions of the cylindrical transducer 302 may vary. A number of elements of the cryoablation system 300 are similar to the one used in cryoablation system 200 such as the catheter 202, lumens 224, 226, and will not be discussed in detail.

Expansion chamber 304 may be an integral part of the catheter 202 or a separate component connected to the distal end of the catheter 202, such as by adhesive bonding or thermal bonding, for example. Chamber 304 may be made of any suitable material expandable or unexpandable in nature, such as polymers. The inlet and exhaust lumens 224, 226 may extend up to the expansion chamber 304 of the catheter 202 encapsulated by the cylindrical transducer 302.

In the embodiment discussed above, the injection of coolant from the inlet lumen 224 may result in vaporization of the liquid in the expansion chamber 304, which in turn cools the transducer 302 and/or the adjacent blood and/or tissue. For example, the coolant may flow from the inlet lumen 224 into the expansion chamber under the transducer 302 in the distal portion of the catheter 202 and back to the fluid withdrawing source 214 via exhaust lumen 226. The cool fluid may absorb or extract heat from the distal portion of the catheter 202, which in turn cools the cylindrical transducer 302 and/or may extract heat from the surrounding tissue and/or blood.

It should be understood that the transducer 210 or 302 may be located at any distance from the expansion chamber, such as the balloon 208, based on the amount of cooling desired. For maximum cooling, the transducer 210 may be positioned directly on the surface of the balloon 208, as shown in FIG. 2, or directly against the expansion chamber 304 in the catheter shaft, as shown in FIG. 3. In other embodiments, the transducer 210, 302 may be disposed at another position close to the balloon 208 or other expansion chamber, such as on the catheter tip or catheter shaft proximate the balloon 208 or other expansion chamber.

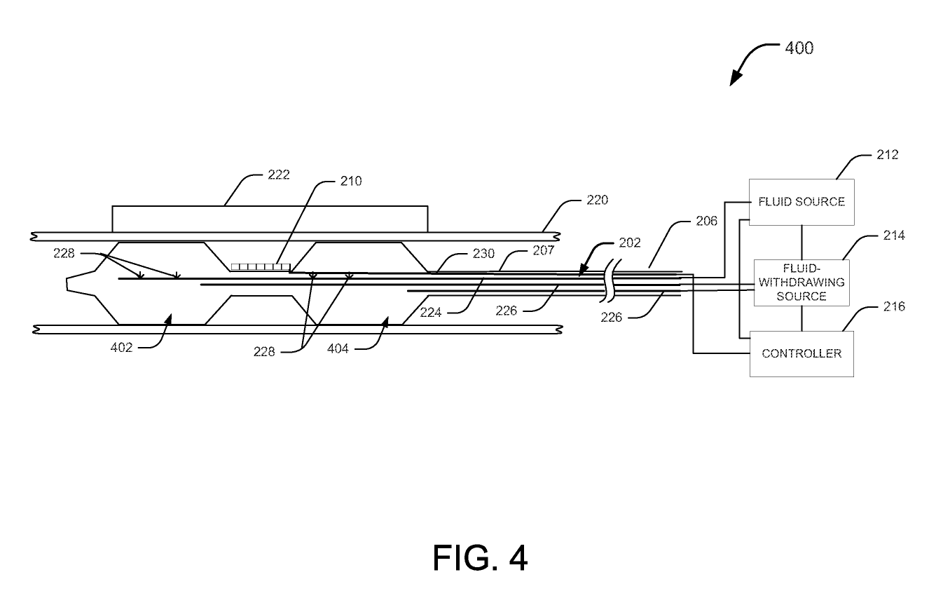

FIG. 4 illustrates an embodiment of a cryoablation system 400 where the catheter 202 may include first and second balloons 402, 404, with a transducer 210 positioned on the catheter shaft at a position between the first and second balloons 402, 404. In this configuration, the inlet lumen 224 may include two sets of orifices 228, each set of orifices 228 positioned within one of the first and second balloons 402, 404. Alternatively, a separate inlet lumen 224 may be provided for each of the first and second balloons 402, 404. Also, to remove expanded gas, each balloon may include a separate exhaust lumen 226, as shown, or the first and second balloons 402, 404 may share an exhaust lumen.

Various alternatives to the cryoablation system 400 may be contemplated. For example, a balloon may be positioned only proximally or distally to the transducer 210. In those embodiments, the balloon may act as a nerve blocker, reducing or preventing transmission of nerve signals that are interpreted as pain. Alternatively, a balloon may be positioned on either side of the transducer, as shown in FIG. 4. The illustrated embodiment may be found particularly useful as a pain blocker as the expandable balloons 402, 404 may be configured to be inflated against a luminal surface of a blood vessel and numb a larger area of the tissue proximate the target tissue 222 to be ablated.

As shown in FIG. 4, the cryoablation system 400 may be configured such that when the balloons 402, 404 are inflated in the vessel such that the outer radial extents of the balloons 402, 404 contact the vessel wall upon inflation, the transducer 210 may remain spaced away from the vessel wall. In other words, the inflated balloons 402, 404 may be configured to be inflated radially outward of the transducer 210. Accordingly, the balloons 402, 404 may be sized such that the transducer 210 is spaced a predetermined distance from the luminal surface of the vessel wall during the ablation process.

Those skilled in the art will understand that the number of transducers positioned for ablation may also vary. For example, instead of a single transducer, as shown in FIGS. 2-4, multiple transducers may be disposed on the balloon, and/or proximally or distally to the balloon 208.

In embodiments using ultrasound transducers, the catheter may be configured such that the fluid is not allowed to boil in front of the transducer, thus may be configured such that the fluid may not change phase to vapor in close proximity to the transducer 210 or 302. Resultant bubbles from the boiling fluid may interfere with the ultrasound beam and may increase heating. To this end, one embodiment of the present disclosure may include an air (or vapor) gap beneath the transducer 210 or 302, spacing the fluid from direct contact with the transducer 210, 302 to prevent propagation of bubbles towards the transducer 210, 302.

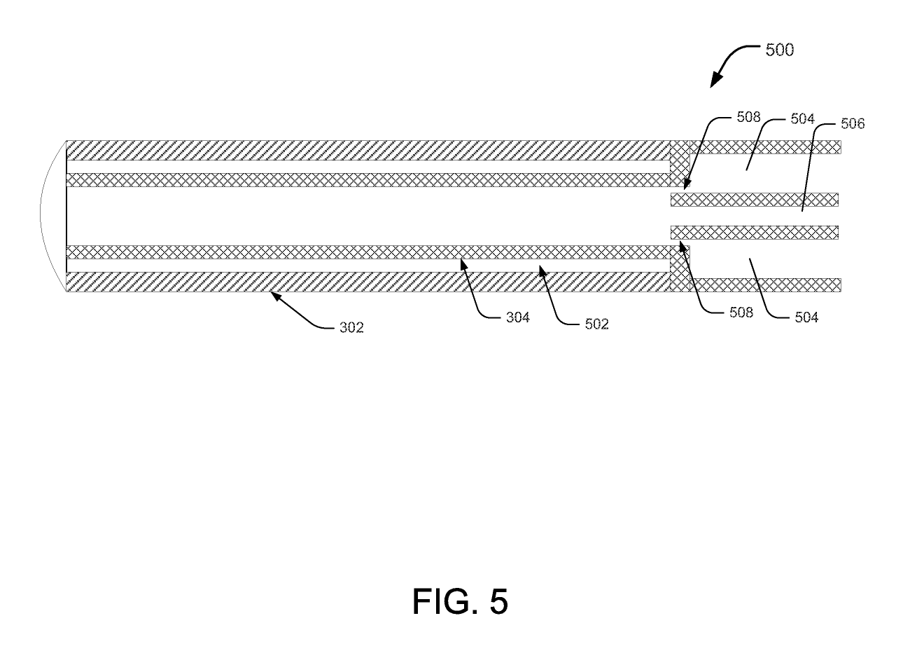

FIG. 5 illustrates an embodiment of the distal end of a cryoablation system 500 including a cylindrical transducer 302 that is fitted over a distal portion of the catheter 202 such that the transducer maintains an air gap 502 with the outer surface of the catheter 202. The air gap 502 may have a low acoustic impedance, creating a large difference in acoustic imdepance with the transducer 302 to reflect/refract sound waves. The width of the air gap 502 may be around 10 to 100 microns, for example. The cryoablation system 500 may also include one or more inlet paths 504 for delivering the fluid to the expansion chamber 304 and outlet paths 506 exhausting fluid from the expansion chamber 304. The inlet path 504 may inject high-pressure liquid to the expandable chamber 304 through orifice 508.

As high-pressure fluid is injected within the chamber 304, the fluid in the chamber 304 may cool and reduce heat from the transducer 302 across the air gap 502. In addition, the air gap 502 may ensure the cooling fluid resides on the side of the transducer that faces the chamber 304 and thus may not interfere with the operation of the transducer 302 and energy emitted from the front side of the transducer 302. In an alternate embodiment, instead of an air gap, a coating or covering to may be suitably employed to protect the transducer 302 from fluid exposure.

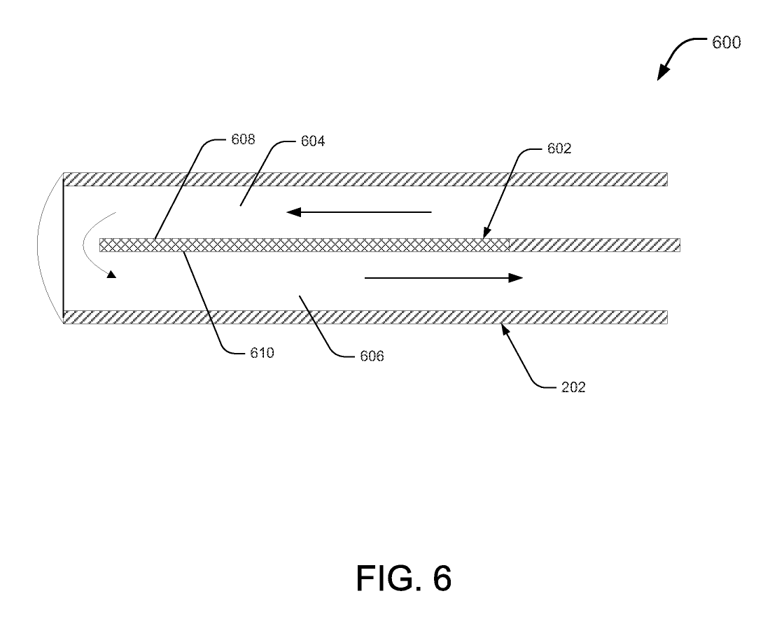

Other techniques to cool the ultrasound transducer effectively may be contemplated without departing from the scope of the present disclosure. In an embodiment of the present disclosure, the transducer 210 may be configured as a disc shaped member positioned within the distal portion of the catheter 202, with the transducer 210 positioned substantially parallel to the longitudinal axis of the catheter 202, as shown in FIG. 6. The transducer 602 may be positioned in catheter 202 such that the transducer 602 divides the interior of the catheter 202 longitudinally into two sections—an inlet section 604 and an outlet section 606. In addition, the transducer 602 includes two faces—front face 608 and rear face 610, with the front face 608 configured to emit ultrasound beams for ablation purposes.

Using the illustrated embodiment, the present disclosure employs a non-boiling fluid, which may be pre cooled prior to reaching the transducer 602. This fluid reaches and passes by the front face 608 of the transducer 602 through the inlet section 604 and returns through the outlet section 606 as the fluid passes by the rear face 610. An example of non-boiling fluid includes, but is not limited to, propane. In some instances, after the fluid passes by the transducer 602 and exits through the exhaust pathway, the exhausted fluid may be partially expanded away from the transducer 602 to subcool additional fluid being delivered toward the transducer 602.

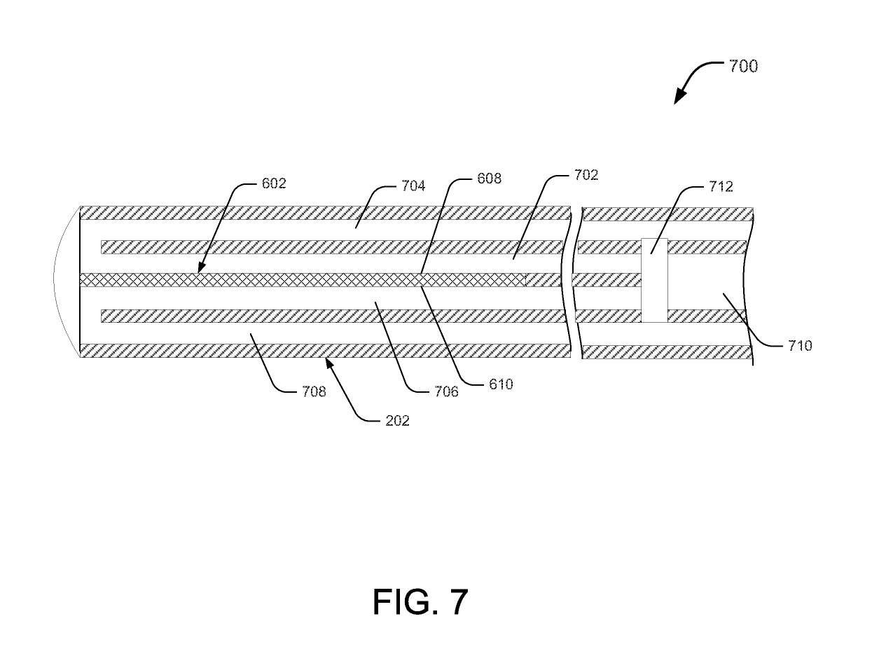

In an alternate embodiment of the present disclosure, fluid in its vapor phase may be used to cool the rear side 610 of the transducer 602, while the front side 608 may be cooled using the fluid in its liquid phase. For example, as shown in FIG. 7, the catheter 202 may include a liquid inlet section 702 and liquid outlet section 704 towards the front side 608 of the transducer 602 for passing a fluid in its liquid phase past the front side 608 of the transducer 602. The catheter 202 may also include a vapor inlet section 706 and a vapor outlet section 708 toward the rear side 610 of the transducer 602 for passing a fluid in its vapor phase past the rear side 610 of the transducer 602. The catheter 202 may also include a fluid inlet 710 and a bubble trap 712 configured to separate a partially expanded fluid into a subcooled liquid portion and a saturated (or super heated) vapor portion.

Using this configuration, the liquid coolant could be partially expanded away from the transducer 602, and the resulting liquid phase may be separated from the gas phase using the bubble trap 712, which is impermeable to the liquid phase, but permeable to the gas phase. Subsequently, the liquid phase coolant may be passed from the fluid inlet 710 towards the front side 608 of the transducer 602 through the liquid inlet section 702, while the vapor is passed towards the rear side 610 of the transducer 602 through the vapor inlet section 706. The liquid and vapor coolant phases may cool the front side and rear side of the transducer 602, respectively, and the expended liquid and vapor coolant may be exhausted from the catheter 202 through the liquid outlet section 704 and vapor outlet section 708.

The following aspects relate to an exemplary method of using the cryoablation system 200 for ablating perivascular renal nerves. For renal ablation therapy, a physician may advance the cryoablation system 200 through the vasculature in a manner known in the art. For example, a guide wire may be introduced percutaneously through a femoral artery and navigated to a renal artery using known techniques, such as radiographic tracking. The catheter 202 may then be introduced into the artery over the guidewire until the distal end of the catheter 202 reaches a desired position proximate the target tissue. In some aspects, the physician may subsequently manipulate the distal portion of the catheter 202 to point towards the target tissue by known steering mechanisms. Once positioned, the distal portion of the catheter 202 including the balloon 208 and the transducer 210 may be located proximate to the target tissue, as shown.

Once the balloon 208 and transducer 210 are positioned proximate the target tissue, coolant may be carried from the fluid source 212 to the balloon 208 through the inlet lumen 224. The fluid may not only act as a coolant, but also may be used to inflate the balloon 208. For example, the fluid delivered through the catheter shaft may inflate the balloon 208 against the luminal wall of the vessel. As the balloon 208 is inflated, the transducer 210, which may be attached to an outer surface of the balloon 208, may also be moved into contact with the luminal wall of the vessel proximate the target tissue, or into a desired spaced relationship with the luminal wall of the vessel. Furthermore, as the fluid enters the balloon 208 via the orifice 228, the fluid may vaporize in the contained region of the balloon 208 acting as an expansion chamber. The illustrated embodiment depicts the balloon 208 in an expanded state such that the transducer 210 is in contact with vessel walls 220, however, in other embodiments the transducer 210 may remain spaced from the vessel wall 220 with the balloon 208 fully expanded.

The phase change of vaporization absorbs considerable heat, which may cool the balloon 208, the transducer 210 and/or blood or tissue surrounding the balloon 208. During this cooling cycle, the coolant, now in a gaseous state, may be returned to the fluid-withdrawing source 214 via exhaust lumen 226. In some instances in which the cooling cycle is a closed system, the fluid-withdrawing source may recycle the gas to the fluid source 212, which may condense the fluid to a liquid to recirculate the fluid. The fluid may flow continuously to and from the balloon 208 using the two lumens 224 and 226, for prolonged cooling. In some embodiments, the cooling mechanism of the present disclosure may cool the balloon 208 in the range of 4 to 30° C. approximately to induce mild hyperthermia to adjacent tissue throughout the tissue ablation process.

The cooling effect provided by the balloon 208 may act as a pain blocker during or before the ablation process, in some instances. In some instances, the phase-changing coolant may cool the expanded balloon 208 to such a degree that the renal nerve tissue in contact with or in close proximately to the inflated balloon may become numb, reducing pain during the procedure. In some situations, the balloon 208 may assist in cooling not just the target tissue, but also the surrounding tissue for pain reduction, arterial spasm, and/or to protect the non-target tissues from thermal damage.

In some instances, the cryoablation system 200 may cool the ultrasound transducer 210 as well as surrounding tissue. For example, the transducer 210 may be positioned proximate to the balloon 208, such as secured to an outer surface of the balloon 208. In an alternate embodiment shown in FIG. 3, the cylindrical transducer 302 encapsulating expansion chamber at the distal portion of the catheter 202 may also provide similar results. Thus, the cryothermal cooling mechanism may conductively extract heat from the transducer 210 during the ablation process. As in FIG. 4, the balloons 402, 404 may cool or numb the tissue of the vessel proximal and/or distal of the target area.

Once the cooling procedure is in place, the transducer 210 may be activated to emit ultrasound energy into the renal tissue 222. During ablation, the cooling provided to the renal tissue and/or the ultrasound transducer 210 may prevent damaging the vascular walls 220 and surrounding tissues and/or allow continuous ablation through an extended duration of time during the ablation process. In addition, cooling the tissue may reduce pain during the ablation process. The sensors 218 disposed proximate the balloon 208 may help monitor the balloon conditions and/or conditions at the target site. For example, a temperature sensor can assist in confirming whether the cooling provided by the balloon 208 is appropriate for the procedure. As a result, the present disclosure provides a simple and cost-effective cooling mechanism to ablate a body tissue without damaging surrounding tissues.

Those skilled in the art will recognize that aspects of the present disclosure may be manifested in a variety of forms other than the specific embodiments described and contemplated herein. Accordingly, departure in form and detail may be made without departing from the scope and spirit of the present disclosure as described in the appended claims.

What is claimed is:

1. A catheter for thermal modulation of renal nerves via thermal ablation, the catheter comprising:
a catheter shaft including a distal end, a proximal end, a first lumen extending therethrough, and a second lumen extending therethrough;
a thermal heating element located proximate the distal end of the catheter shaft; and
a phase-change cooling mechanism configured to extract heat away from the thermal heating element, the phase-change cooling mechanism extracting heat through vaporization of a liquid refrigerant circulated to the distal end of the catheter shaft through the first lumen into a gas and exhausting the gas to the proximal end of the catheter shaft through the second lumen.

2. The catheter of claim 1, wherein the catheter includes a balloon mounted to the distal end of the catheter shaft, the liquid refrigerant being circulated to an interior of the balloon.

3. The catheter of claim 2, further comprising an evaporator located within the balloon to vaporize the liquid refrigerant.

4. The catheter of claim 2, wherein the thermal heating element is mounted on the balloon.

5. The catheter of claim 1, wherein the thermal heating element is an ultrasound transducer.

6. The catheter of claim 1, wherein the liquid refrigerant is circulated to the distal end of the catheter shaft at a first pressure, and the exhausted gas is circulated from the distal end of the catheter shaft at a second pressure, less than the first pressure.

7. The catheter of claim 1, wherein the liquid refrigerant expands through one or more orifices positioned proximate the distal end of the catheter.

8. The catheter of claim 7, wherein the one or more orifices are positioned within an expansion chamber mounted to the distal end of the catheter.

9. The catheter of claim 1, wherein the phase-change cooling mechanism is configured to extract heat directly from the thermal heating element.

10. A catheter for thermal ablation of renal nerve tissue, the catheter comprising:
a catheter shaft having a proximal end and a distal end;
an ultrasound transducer positioned proximate the distal end of the catheter shaft configured to thermally heat body tissue; and
a phase-change cooling mechanism configured to extract heat away from the ultrasound transducer, the phase-change cooling mechanism including an expansion chamber positioned proximate the distal end of the catheter shaft configured to convert a liquid refrigerant circulated to the expansion chamber through the catheter shaft into a gas.

11. The catheter of claim 10, wherein the ultrasound transducer is positioned on the expansion chamber.

12. The catheter of claim 11, wherein the expansion chamber is a balloon mounted on the distal end of the catheter shaft.

13. The catheter of claim 10, wherein the liquid refrigerant expands through one or more orifices located within the expansion chamber.

14. The catheter of claim 10, wherein the phase-change cooling mechanism is configured to extract heat directly from the ultrasound transducer.

15. The catheter of claim 10, wherein the ultrasound transducer surrounds the expansion chamber.

16. A method of intravascularly thermally modulating renal nerve tissue, the method comprising:
advancing a catheter through a vessel such that a distal end of the catheter is positioned proximate a target location;
activating a thermal heating element mounted on the distal end of the catheter to thermally heat a renal nerve tissue; and
cooling the thermal heating element with a phase-change cooling mechanism, comprising:
i) delivering a pressurized liquid refrigerant through the catheter to an expansion chamber proximate the thermal heating element;
ii) expanding the pressurized liquid refrigerant into a gas to absorb heat dissipated from the thermal heating element; and
iii) exhausting the gas from the expansion chamber.

17. The method of claim 16, wherein the expansion chamber includes a balloon secured to the distal end of the catheter.

18. The method of claim 17, wherein the liquid refrigerant expands through one or more orifices positioned within the balloon.

19. The method of claim 16, wherein the thermal heating element is an ultrasound transducer.

20. The method of claim 19, wherein the gas absorbs heat directly from a surface of the ultrasound transducer.
